(19) United States
(12) Patent Application Publication
(10) Pub. No.: US 2012/0041384 A1
(43) Pub. Date: Feb. 16, 2012

(54) SINGLE USE DEVICE FOR DELIVERY OF CARTRIDGE DRUGS

(75) Inventors: Melvin A. Finke, DeLand, FL (US); David Selvitelli, Suffield, CT (US); Kathleen Tremblay, Westfield, MA (US); John K. Foster, Port Orange, FL (US); Carl Strickland, DeLand, FL (US)

(73) Assignee: TYCO HEALTHCARE GROUP LP, Mansfield, MA (US)

(21) Appl. No.: 13/208,408

(22) Filed: Aug. 12, 2011

Related U.S. Application Data
(60) Provisional application No. 61/373,164, filed on Aug. 12, 2010.
(60) Provisional application No. 61/471,909, filed on Apr. 5, 2011.

(51) Int. Cl. A61M 5/315; A61M 5/32; A61M 5/31

(52) U.S. Cl. 604/194; 604/227; 604/200; 604/201

(57) ABSTRACT

A syringe for administering medicament from a cartridge. The syringe includes a sleeve having an interior for receiving the cartridge extending between a distal hub and a proximal opening. The syringe has a cap selectively covering the proximal opening for retaining the cartridge in the interior of the sleeve. The cap includes a finger grip extending outward from the sleeve for engaging fingers of a user. The syringe also has a plunger rod movably connected to the cap. The plunger rod has a distal connector for selectively connecting the rod to the cartridge plunger when the cartridge is received in the interior of the sleeve to push medicament from the cartridge reservoir as the plunger rod moves with respect to the cap.

### CROSS REFERENCE TO RELATED APPLICATION

This application claims priority to U.S. Patent Application Ser. No. 61/373,164, filed Aug. 12, 2010, entitled, “Single Use Device for Delivery of Cartridge Drugs”, and to U.S. Patent Application Ser. No. 61/471,909, filed Apr. 5, 2011, entitled, Medical Cartridge Receiver Having Access Device, which are hereby incorporated by reference in their entireties for all purposes.

### BACKGROUND

The present disclosure relates generally to devices for injection of liquid medicaments that are prefilled cartridges and, in particular, the present disclosure is directed to a “ready to use/ready to dispose” devices for single use injection of local anesthetics.

Local anesthetic is frequently used to numb tissue in a patient's mouth to reduce pain and discomfort a patient may feel during a dental procedure. Conventionally, a reusable syringe assembly is used to inject the anesthetic or medicine from a cartridge. The syringe assembly includes a barrel for receiving a cartridge, a plunger rod or harpoon slidably received in a head of the barrel for actuating the cartridge, and a delivery needle connected to a hub of the barrel for delivering the anesthetic to the patient. The syringe may be constructed of chrome plated brass and stainless steel and may include a needle adapter, syringe barrel, plunger rod with harpoon, finger grip and a thumb ring. Typically, the syringe must be sterilized between each use.

The needle permits local anesthetic to travel from the dental cartridge into tissue surrounding the needle tip. Needles may be pre-sterilized and disposable. The needle may be composed of a single piece of metal tubing around which is placed a plastic or a metal syringe adapter and a needle hub which is attached to the needle adapter of the syringe.

The cartridge or carpule is a glass cylinder containing a local anesthetic among other ingredients. A diaphragm at one end of the cylinder is held in place by an aluminum band. The opposite end of the cylinder includes a moveable piston or stopper. Typically, the diaphragm of the cartridge is swabbed with alcohol before being loaded into a pre-sterilized syringe. As the cartridge is loaded into the syringe, an access needle at the hub of the barrel pierces the cartridge diaphragm so the anesthetic in the cartridge can be dispensed. The plunger rod of the syringe pushes the piston of the cartridge toward the diaphragm to force the anesthetic through the access needle and ultimately into the delivery needle through which it is delivered to the patient's tissue.

Assembly of a dental syringe often requires removing a sterilized syringe from its container and placing an alcohol wiped cartridge into the syringe with the plunger rod of the syringe fully retracted. A rubber septum supported on a distal end of the syringe end of the cartridge is inserted into the syringe first and a harpoon or like structure of the plunger rod is engaged with a plunger positioned at a proximal end of the cartridge. A needle assembly including a needle and protective cap can be secured to the syringe. The protective cap is then removed from the needle and the syringe is ready for use. As will be appreciated by those skilled in the art, between each use the syringe must be sterilized, a new delivery needle must be mounted on the syringe, and a new cartridge must be prepared and loaded.

After use, the needle must be recapped, disconnected from the syringe and disposed in a sharps container and the cartridge must be removed and disposed in a sharps container. Afterwards, the syringe must be transported for sterilization processing. These multi-step assembly and post-use procedures take time and are resource and labor intensive. There is a need for a syringe assembly that reduces the time, resources, and labor required to provide anesthetic to a patient.

### SUMMARY

In one aspect, the present invention includes a syringe for administering medicament from a cartridge having a medicament reservoir and a plunger mounted in the reservoir for pushing medicament from the reservoir. The syringe comprises a sleeve having an interior sized and shaped for receiving the cartridge. The interior extends between a distal hub and a proximal opening opposite the hub sized and shaped for delivering the cartridge to the interior. The syringe also includes a cap permanently affixed to and covering the proximal opening for retaining the cartridge in the interior of the sleeve. The cap includes a finger grip extending outward from the sleeve for engaging fingers of a user administering medicament when the cap covers the proximal opening. Further, the syringe comprises a plunger rod movably connected to the cap. The plunger rod has a distal connector for connecting the rod to the cartridge plunger when the cartridge is received in the interior of the sleeve to push medicament from the cartridge reservoir as the plunger rod moves with respect to the cap.

In another aspect of the invention, a syringe for administering medicament from a cartridge comprises a sleeve having an interior sized and shaped for receiving the cartridge extending between a distal hub and a proximal opening opposite the hub sized and shaped for delivering the cartridge to the interior. The syringe also comprises a cartridge having a medicament reservoir and a plunger, the cartridge disposed within the sleeve and a cap affixed to the sleeve and covering the proximal opening for retaining the cartridge in the interior of the sleeve, the cap including a finger grip extending outward from the sleeve for engaging fingers of a user administering medicament. A plunger rod may be movably connected to the cap, the plunger rod having a distal connector for connecting the rod to the cartridge plunger when the cartridge is received in the interior of the sleeve to push medicament from the cartridge reservoir as the plunger rod moves with respect to the cap. The syringe may also comprise a catheter having a proximal end fluidly connected to the distal hub of the sleeve and a distal end fluidly connected to a needle.

In another aspect, the present invention includes a syringe comprising a sleeve having an interior sized and shaped for receiving the cartridge extending between a distal hub and a proximal opening opposite the hub sized and shaped for delivering the cartridge to and from the interior. The syringe includes a needle extending from the distal hub for piercing a patient and a plunger rod removably covering the needle to prevent needle sticks. The plunger rod is removable from the needle and selectively connectable to the cartridge plunger when the cartridge is received in the interior of the sleeve to push medicament from the cartridge reservoir as the plunger rod moves with respect to the cap.

In yet another aspect, the present invention includes a syringe comprising a sleeve having an interior sized and shaped for receiving the cartridge extending between a distal hub and a proximal opening opposite the hub sized and shaped for delivering the cartridge to and from the interior. The syringe also comprises means for limiting movement of the cartridge towards the proximal opening in the sleeve. The syringe also includes a needle extending into the sleeve from the distal hub for piercing the cartridge to access medicament in the cartridge reservoir and a plunger rod movably connected to the sleeve. The plunger rod has a distal connector for selectively connecting the rod to the cartridge plunger when the cartridge is received in the interior of the sleeve to push medicament from the cartridge reservoir as the plunger rod moves with respect to the sleeve. The plunger rod is adapted to move the plunger of the cartridge between a first position, in which the septum is not pierced by the needle and the plunger of the cartridge is in a proximal location, and a second position, in which the septum is pierced by the needle and the plunger of the cartridge is in a distal location.

In still another aspect, the present invention includes a syringe comprising a sleeve having an interior sized and shaped for receiving the cartridge extending between a distal hub and a proximal opening opposite the hub sized and shaped for delivering the cartridge to and from the interior. The syringe further comprises a needle extending from the distal hub for piercing a patient and a flange including an inner annular ring adapted to engage a proximal end of the cartridge and a ratchet protrusion permitting the flange to move distally with respect to the sleeve and preventing the flange from moving proximally with respect to the sleeve so the ratchet protrusion retains the cartridge in a pre-armed state, in which the needle does not pierce the septum, before the flange is moved distally with respect to the sleeve and the ratchet protrusion prevents proximal movement of the flange with respect to the sleeve and retains the cartridge in an armed state, in which the needle pierces the septum, after the flange is moved distally with respect to the sleeve. Further, the syringe includes a plunger rod movably connected to the finger flange. The plunger rod has a distal connector for selectively connecting the rod to the cartridge plunger when the cartridge is received in the interior of the sleeve to push medicament from the cartridge reservoir as the plunger rod moves with respect to the sleeve.

In another aspect, the present invention includes a syringe comprising a sleeve having an interior sized and shaped for receiving the cartridge extending between a distal end and a proximal end opposite the distal end. The syringe also includes a plunger rod movably connected to the sleeve. The plunger rod has a distal connector for selectively connecting the rod to the cartridge plunger when the cartridge is received in the interior of the sleeve to push medicament from the cartridge reservoir as the plunger rod moves with respect to the sleeve. In addition, the syringe comprises a hub moveably attached to the distal end of the sleeve. The hub includes a needle extending into the sleeve and is movable between a first position, in which the septum is not pierced by the needle, and a second position, in which the septum is pierced by the needle.

In yet another aspect, the present invention includes a syringe comprising a sleeve having an interior sized and shaped for receiving the cartridge extending between a distal hub and a proximal opening opposite the hub sized and shaped for delivering the cartridge to and from the interior. The syringe also has a needle extending into the sleeve and a removable barrier positioned between the needle and the cartridge when received in the sleeve to prevent the needle from piercing the septum of the cartridge. Removal of the barrier permits the needle to pierce the septum to permit flow of medicament from the cartridge through the needle.

In another aspect, the present invention includes a syringe comprising a sleeve having a medicament reservoir, a plunger rod, and a plunger. Further, the syringe includes a needle extending into the sleeve and a thumb slide movably attached to the sleeve and adapted to engage the plunger rod when received in the sleeve to move the cartridge plunger distally and/or proximally to permit flow of medicament from the cartridge through the needle.

Other aspects of the present invention will be apparent in view of the following description and claims.

### BRIEF DESCRIPTION OF THE DRAWINGS

FIG. 1A is a perspective of a single use syringe assembly of a first embodiment of the present invention;

FIG. 1B is a separated view of the single use syringe assembly;

FIG. 1C is a sectional view of a cartridge of the single use syringe assembly;

FIG. 1D is a perspective of the single use syringe in a depressed position;

FIGS. 2A and 2B are perspectives of a single use syringe of a second embodiment of the present invention;

FIG. 2C is a sectional view of a rubber plunger and rod of the single use syringe of the second embodiment;

FIG. 3 is a perspective of a single use syringe of a third embodiment;

FIGS. 4A and 4B are perspectives of a single use syringe of a fourth embodiment;

FIG. 5 is a perspective of a single use syringe of a fifth embodiment;

FIG. 6 is a perspective of a single use syringe of a sixth embodiment;

FIG. 7A is a partially separated section of a single use syringe of a seventh embodiment;

FIG. 7B is a side elevation of a plunger assembly of an alternative embodiment;

FIGS. 8A and 8B are sections of a single use syringe of another embodiment;

FIGS. 9A and 9B are sections of a needle hub of yet another embodiment;

FIGS. 10A-10D are views of a needle hub of another embodiment;

FIGS. 11A-11C are various views of a finger hold of another embodiment;

FIG. 12 is a perspective of a single use syringe of yet another embodiment;

FIGS. 13 and 14 are sections of a plunger assembly of another embodiment;

FIGS. 15A and 15B are side elevations of a single use syringe of other embodiments; and

FIG. 16 is a perspective of a needle assembly of yet another embodiment.

Other aspects of the present invention will be apparent in view of the following description and claims.

### DETAILED DESCRIPTION OF THE EMBODIMENTS

Particular embodiments of the present disclosure are described hereinbelow with reference to the accompanying drawings; however, the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

In the discussion that follows, the term “proximal” or “trailing” will refer to the portion of a structure that is closer to a clinician, while the term “distal” or “leading” will refer to the portion of the structure that is farther from the clinician. As used herein, the term “subject” refers to a human patient or other animal. The term “clinician” refers to any medical personnel including a dentist, dental hygienist or other care provider and may include support personnel. The term “cartridge” refers to a removable structure such as a carpule, canister or the like for housing a liquid medicament such as a local anesthetic or the like.

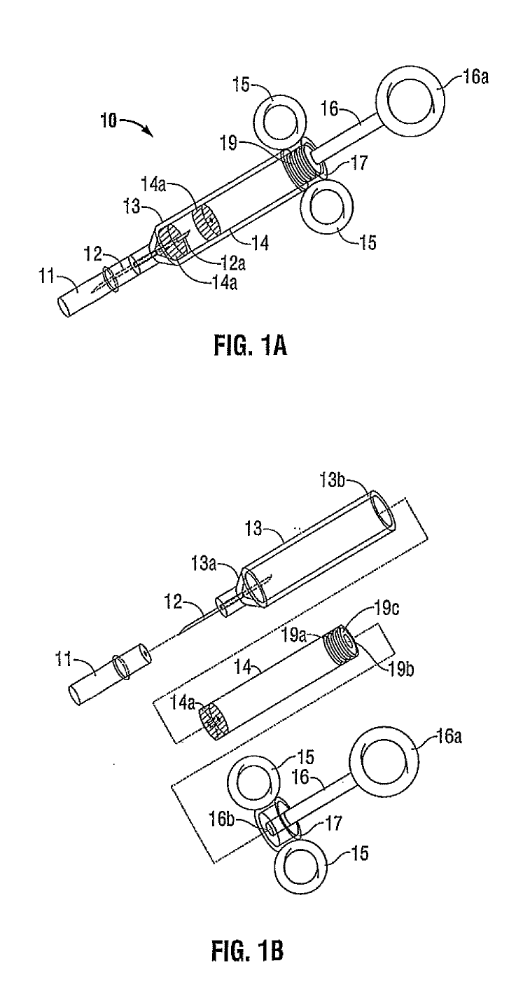

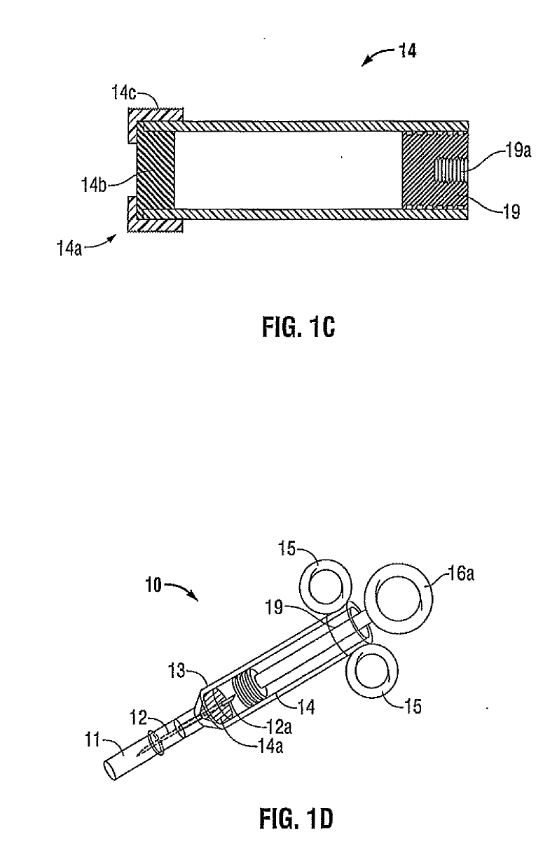

Referring to FIGS. 1A-1D, a single use syringe of a first embodiment of the present invention is designated in its entirety by the reference number 10. The syringe 10 includes a sleeve 13 having a distal hub 13a and a proximal opening 13b opposite the hub. The hub 13a has a staked needle 12. The staked needle 12 may be a double tipped needle, having one tip extending distally from the hub 13a to insert into tissue of a patient and the other tip extending proximally into an interior of the sleeve 13 to pierce a septum 14a of a cartridge 14. A removable cover 11 is fitted over the needle 12 to prevent accidental needle sticks prior to use of the syringe 10. A prefilled cartridge 14 is positioned in the interior of the sleeve 13 by passing the cartridge through the proximal opening 13b of the sleeve.

The cartridge 14 includes a movable plunger 19 formed from a conventional material, such as rubber or synthetic elastomer. The plunger 19 has a distal face 19a for contacting a liquid medicament located in a reservoir inside the cartridge 14. The plunger 19 also has a proximal face 19b that engages a distal end 16b of a plunger rod 16. The distal end 16b of the plunger rod 16 may comprise a connector such as a barb to harpoon the plunger 19 so the plunger may be drawn proximally to aspirate the cartridge 14. Alternatively, the proximal face 19b of the plunger 19 may include a connector 19c for receiving the distal end 16b of the plunger rod 16. The connector 19c may include a protrusion or an orifice sized and shaped to engage a complementary sized and shaped portion of the distal end 16b of the plunger rod 16. In one embodiment shown in FIG. 1B, the connector 19c includes an opening configured to receive the distal end 16b of the plunger rod 16. In other embodiments, the connector 10c and distal end 16b included threaded fasteners. A distal end 14a of cartridge 14 includes a septum 14b formed of conventional materials, such as rubber or synthetic elastomer forming a fluid tight seal. In one embodiment shown in FIG. 1C, the septum 14b may be secured to the cartridge 14 by an aluminum band 14c crimped around the distal end 14a of the cartridge 14.

A plunger assembly is removably or permanently attached to the proximal opening 13b of the sleeve 13 and may include the plunger rod 16, a finger grip 15, and a cap 17. The plunger rod 16 may have a thumb ring 16a at its proximal end. The finger grip 15 may be of any size and shape to provide a surface sufficient to support one or more fingers during use. In the embodiment shown in FIG. 1b, the finger grip 15 comprises a pair of finger rings, each positioned on an opposite side of the cap 17. The cap 17 may be removably attached to the proximal opening 13b of the sleeve 13 in a conventional manner, such as by screwing the cap onto the sleeve, or by an interference fit. In another embodiment, the cap 17 is permanently affixed to the proximal opening to maintain the cartridge within the sleeve, so as to prevent or reduce release of medicament and/or safety concerns should the syringe be dropped or otherwise damaged resulting in breakage of a glass cartridge within the sleeve. Delivering the syringe/cartridge to the user preassembled so that the cartridge is secured within the sleeve may ensure sterility of the fluid pathway. The preassembled syringe/cartridge may also eliminate the need of wiping the cartridge cap with a disinfectant prior to assembly by the clinician and may also eliminate inadvertent bending of the needle which may occur when the clinician inserts the cartridge into the sleeve.

To assembly the syringe 10, a user inserts a cartridge 14 into the interior of the sleeve 13 through the proximal opening 13b so the septum 14b faces the distal hub 13a. The cap 17 is attached to the sleeve 13 so it covers the proximal opening 13b. In some embodiments, the cartridge 14 and the syringe 10 may be delivered to the user once the cap 17 is secured to the sleeve 13 without engaging the plunger rod 16 and the plunger 19. In these embodiments, the user moves the plunger rod 16 distally using the thumb ring 16a to engage at least a portion of distal end 16b of the plunger rod 16 with the connector 19c of the plunger 19 positioned in the cartridge 14. Alternatively, the plunger rod 16 may be moved distally to engage the distal end 16b of the plunger rod with the connector 19c on the plunger 19 before the cartridge 14 and the syringe 10 are delivered to the user. Regardless, the user moves the cartridge 14 distally by pushing the plunger rod 16 distally until the proximal end 12a of the needle 12 pierces the septum 14b of the cartridge 14, allowing to the medicament to flow through the needle. Before inserting the needle 12 into the patient, the user removes the cap 11 to expose the distal end of the needle 12. The user may also push the plunger rod 16 distally until a few drops of medicament are released from the needle 12. Once the needle 12 is inserted in the patient, the plunger rod 16 is pushed distally to advance the plunger 19 in the interior the cartridge 14, as shown in FIG. 1D, so medicament is delivered through the needle 12 to a patient. After the local anesthetic is delivered to the patient, the entire syringe 10 may be placed in a sharps container, or the plunger assembly may be removed for reuse before placing the sleeve 13 and the cartridge 14 in the sharps container.

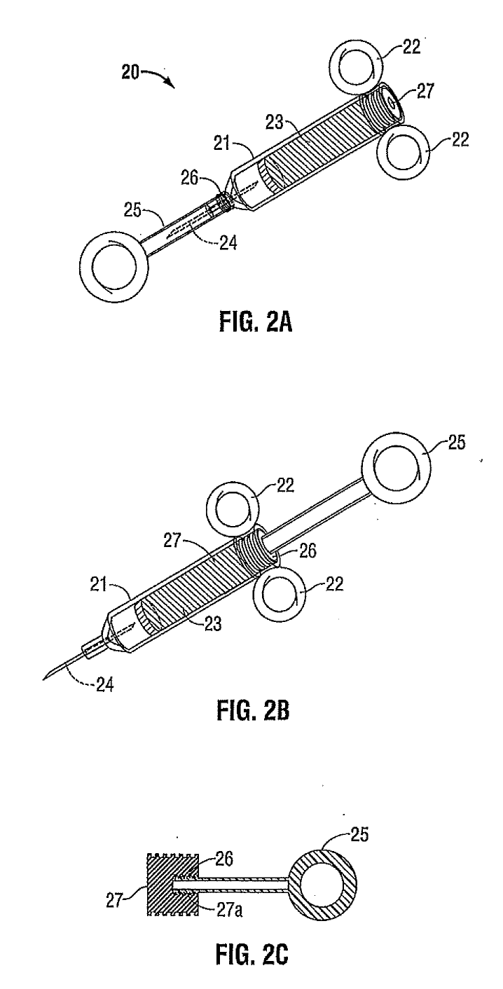

Referring now to FIGS. 2A-2C, a single use syringe according to a second embodiment of the present invention is designated in its entirety by the reference number 20. As shown in FIGS. 2A and 2B, the syringe 20 includes a sleeve 21 with a staked needle 24, finger grips 22 and a cartridge 23 with a plunger 27. The components of the syringe 20 are similar to the corresponding components of the syringe 10 described above, except that instead of a cap 11 as shown in FIGS. 1A and 1B, a partially hollow plunger rod 25 is provided and acts as a protective sheath for the needle 24. The plunger rod 25 may be removably attached to the distal end of the sleeve 21 by conventional means, such as with an interference fit, cooperative threading, and adhesives. The plunger rod 25 may be removed from the needle 24 and attached to the plunger 27 at the proximal end of the sleeve 21. Threads 26 may be provided at the end of the plunger rod 25 for attachment to the rubber plunger 27 and for attachment to the distal end of the sleeve 21. The presence of a thumb ring on plunger rod 25 may provide an additional surface for the user to generate sufficient torque to easily detach the plunger rod 25 from the distal end of the sleeve. As shown in FIG. 2C, the threads 26 engage corresponding threads 27a in the plunger 27. Although FIG. 2C shows threads 26 on an outer diameter of the plunger rod 25 and corresponding threads 27a on an inner diameter of the rubber plunger 27, the plunger rod may be modified so that threads 26 are disposed on an inner diameter of plunger rod 25, while corresponding threads 27a may be disposed on an outer diameter of the rubber plunger. Alternatively, other known engagement techniques may be used to secure the plunger rod 25 to the plunger 27 and about the needle 24.

In another embodiment (not shown), an assembly comprising a plunger rod and finger grips may be removably attached to the distal end of the sleeve so that the plunger rod covers the needle extending. When the plunger rod and finger grip assembly is removed from the distal end of the sleeve, the assembly may be removably attached to the proximal end of the sleeve to allow the plunger rod to engage the plunger in the cartridge. The plunger rod may engage the plunger via a connector on the proximal face of the plunger, it may pierce the proximal face of the plunger, or, in a system comprising a self-aspirating needle, it may abut the proximal face of the plunger. The plunger rod and finger grip assembly may be removably attached to either the distal or proximal end of the sleeve by any conventional mounting structure, such as a friction fit structure or screw threads. Though not necessary, in one embodiment the distal end and the proximal end of the sleeve comprise the same mounting structure.

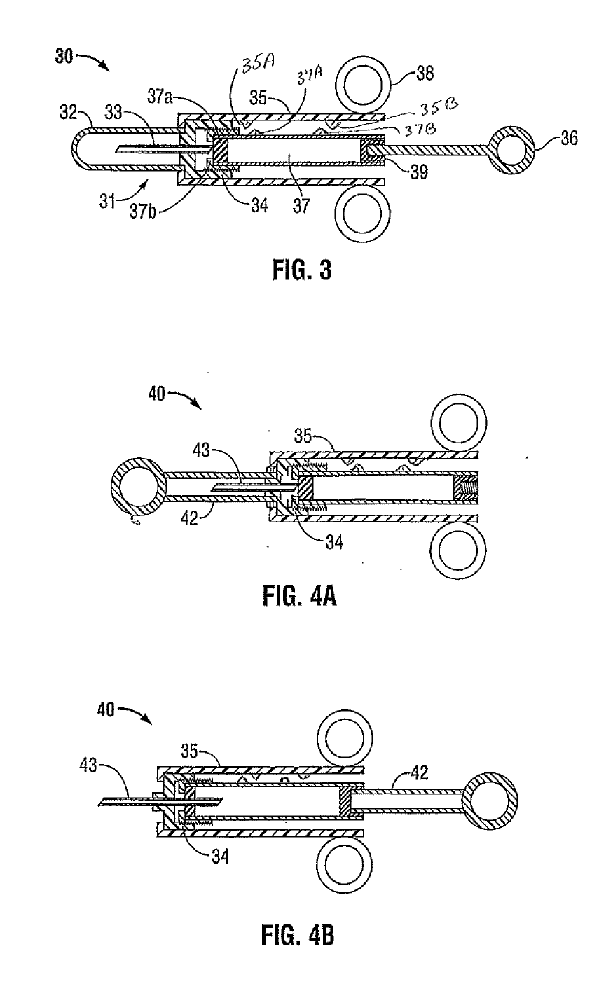

As shown in FIG. 3, syringe 30 of a third embodiment includes a cartridge 37 having a movable plunger 39, a septum 37a, and a cap 37b. The plunger, septum, and cap of the syringe 30 are similar to those shown in FIGS. 1A and 1B and may be made from conventional materials. The syringe 30 also includes a separate transfer needle 31 having a needle 33 and a hub 34. The proximal end of the hub 34 may include a sterile cover (not shown) made from foil or Tyvek sheet that is removed prior to attaching the transfer needle 31 to the cartridge 37. Tyvek is a US federally registered trademark of the E. I. du Pont de Nemours and Company. Alternatively, the sterile cover may remain in place while attaching the transfer needle 31 to the cartridge 37 so the needle 31 pierces the sterile cover. The hub 34 of the transfer needle 31 may be removably coupled to the distal hub of the cartridge 37 in a conventional manner, e.g., by screwing the transfer needle 31 onto a luer lock fitting on the cartridge 37. A cover 32 covers the needle 33. A sleeve 35 snaps over or around the cartridge 37. The sleeve 35 may also snap over or around the transfer needle 31 and the cartridge 37 (not shown). The sleeve 35 may be made of conventional material. In one embodiment, the sleeve 35 may be formed of plastic or cardboard and may include finger grips 38. A plunger rod 36 may be attached to the rubber plunger 39 in a manner previously described. Detents 35A on an inside surface of the sleeve may cooperate with detents 37A on an outside surface of the cartridge to prevent inadvertent arming of the cartridge. Similarly, detents 35B on an inside surface of the sleeve may cooperate with detents 37B on an outside surface of the cartridge to maintain the cartridge within the sleeve. It is understood that one or more recesses may replace one or more detents, so long as there is cooperative engagement between the detents or recesses on the sleeve and the recess or detents on the cartrdige.

As illustrated in FIGS. 4A and 4B, a single use syringe 40 of a fourth embodiment is substantially similar to the syringe 30 of the third embodiment. The syringe 40 includes a plunger rod 42 that also functions as a protective cover for the needle 43 prior to using the syringe 40 instead of including a separate cover 32 as in the syringe 30 of the third embodiment. The plunger rod 42 is removed from the distal end of the syringe 40 and attached to the rubber plunger 49 in the cartridge 47. As shown in FIG. 4A, a needle hub 44 is constructed and arranged to engage the outer surface of the distal end of the sleeve 45. Alternatively, the needle hub 44 can be formed so it engages a portion of the sleeve 45 surrounding the needle 43, to avoid an increase in diameter associated with engaging the outer surface of the sleeve. One or more detents or recess on an inner surface of the sleeve may cooperate with one or more recess or detents on an outer surface of the cartridge to prevent inadvertent arming and or to maintain the cartridge within the sleeve, as also described with respect to FIG. 3.

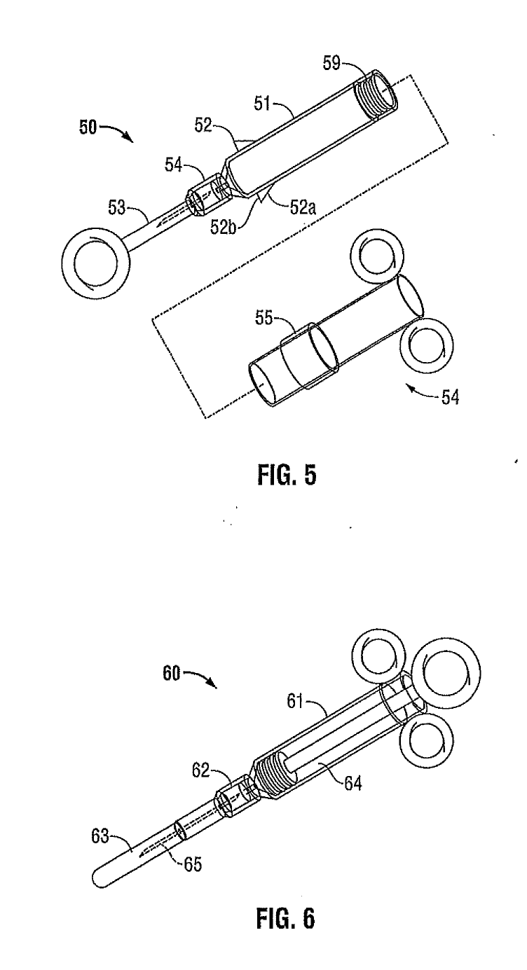

FIG. 5 depicts a single use syringe 50 according to the fifth embodiment of the present invention. As shown in FIG. 5, the syringe 50 includes a cartridge 51 attached to a transfer needle 54 as described above with regard to FIG. 3. The cartridge 51 includes a locking structure 52, which is shown near the distal end of the cartridge but may be disposed anywhere on the cartridge. The locking structure 52 may be annularly positioned about the cartridge 51 or, alternatively, may include one or more engagement members. A sleeve 54 is provided and includes a lock ring 55 that may slide along the sleeve to engage the locking structure 52. The locking structure 52 may have an angled proximal surface 52a to facilitate attaching the sleeve 54 to the cartridge 51, and a substantially perpendicular distal face 52b to prevent separation of the sleeve from the cartridge. As described above in previous embodiments, a needle cover 53 may be used as a plunger rod and attached to the proximal face of the rubber plunger 59 of the cartridge 51.

As shown in FIG. 6, a sixth embodiment of a single use syringe 60 is similar to the single use syringe 10 shown in FIG. 1. The syringe 60 has a sleeve 61 in which a cartridge 64 may be disposed. Unlike the syringe 10 of the first embodiment, having a staked needle 12, the syringe 60 of the sixth embodiment uses a transfer needle assembly 62 that is releasably secured to a distal end of the sleeve 61, e.g., by a luer connector. A cover 63 may be provided on the transfer needle assembly 62 to enclose a needle 65 of the transfer needle assembly 62 before and after use.

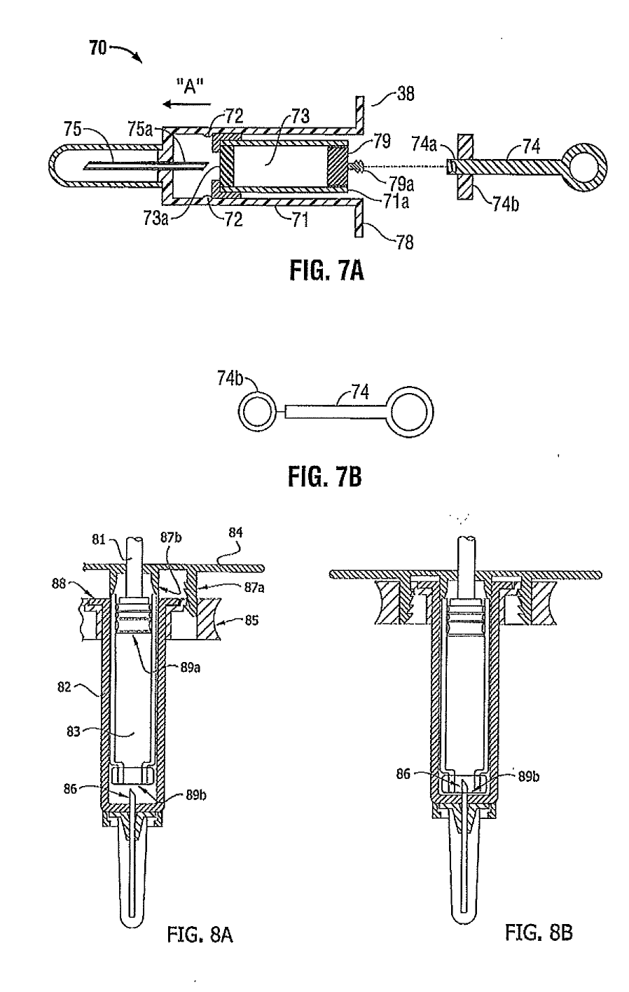

FIG. 7 depicts a single use syringe 70 according to a seventh embodiment of the present invention. The features described below with respect to FIG. 7 may be included in any of the single use syringes described throughout this application. The syringe 70 includes a sleeve 71 having a staked needle 75 that accommodates a cartridge 73.

The sleeve 71 may include an injection molded syringe barrel with an integral finger flange 78 and the staked needle 75. The standard cartridge 73 is loaded into the sleeve 71 and an injection molded ring plunger rod 74 is attached to a proximal end 71a of the sleeve 71 behind the cartridge 73 to lock the cartridge inside the sleeve 71. A distal end 74a of the plunger 74 may have internal threads that accept a threaded rod 79a projecting from the proximal face of the cartridge plunger 79. Alternatively, the plunger 74 may have a barb or harpoon (not shown) on its distal end that engages the cartridge plunger 79. The sleeve, and/or the cartridge may include means for limiting the movement of the cartridge towards proximal opening of the sleeve. The limiting means may include one or more protrusions or recesses on the interior of the sleeve which cooperate with one or more recesses or protrusions on the exterior of the cartridge.

Alternatively, the plunger 74 may include a “break-away” ring 74b at its distal end, as shown in FIG. 7B. The ring 74b is press-fit into the inner diameter of the proximal end 71a of the sleeve 71 and engages the cartridge 73 to lock it in the sleeve thereby limiting movement of the cartridge towards the opening of the sleeve. The plunger 74 and the ring 74b are twisted to break the plunger from the ring, and the plunger can then be attached to the cartridge plunger 79.

The syringe 70 may include an annular ridge or detent 72 located inside the sleeve 71 near the distal end of the syringe. The ring or detent 72 spaces the cartridge 73 from the needle 75 to prevent the needle from penetrating the septum 73a of the cartridge before the syringe 70 is ready to use. Once the user secures the plunger rod 74 to the cartridge plunger 79, the user advances the cartridge 73 past the detent 72 by pressing the plunger rod 74 distally in direction “A” as shown in FIG. 7A until the cartridge engages the hub of the syringe 70 so the syringe is armed. Arming occurs when the proximal end 75a of the needle 75 pierces the cartridge septum 73a. The detent 72 may also be configured to engage an outer surface of the cartridge 73 to prevent the cartridge from becoming dislodged once armed.

In an alternative embodiment (not shown), the cartridge 73 may include one or more annular recesses. The cartridge 73 may comprise two annular recesses around the distal end of the cartridge. The first recess may be formed about a cap and the second recess may be formed where the cap and the cartridge 73 connect. The first recess may cooperate with the detent 72 to keep the cartridge 73 a predetermined distance from the plunger rod 74 and keep the cartridge in the sleeve 71 without a plunger rod attached to the sleeve. To arm the cartridge 73, the plunger rod 74 is depressed, moving the first annular recess past the detent 72 and moving the second annular recess into cooperation with the detent 72. The cooperation of the detent 72 and the second recess of the cartridge 73 keeps the cartridge in the sleeve 71 without a plunger being attached to the sleeve.

The syringe 70 may be provided to the user preassembled and ready to use. When the cartridge 73 is emptied, the entire device is discarded, eliminating the need to remove the cartridge from the sleeve 71, recap the needle 75 and reload a second cartridge 73. As a result, the syringe 70 saves time and prevents risk of needle stick when the cartridge is removed and loading. Further, the manufacturing the syringe 70 is more efficient because fewer injection molded components are used, thereby providing a low part count assembly which may result in faster assembly times and lower costs. The syringe 70 may be molded from clear plastic that encapsulates the glass cartridge 73 in case the glass cartridge breaks when over pressurized. Additionally, the sleeve 71 and the plunger rod 74 provide a sterile seal around the cartridge 73, enabling the syringe 70 to be assembled in a clean room.

FIGS. 8A and 8B depict a single use syringe 80 according to another embodiment. The syringe 80 shown in FIGS. 8A and 8B lengthens a standard 3 mL syringe 82 to accommodate a standard 1.8 mL cartridge 83. The syringe 82 includes a needle 86 that may be a staked needle or a separate transfer needle. An assembly including finger flanges 84, a plunger rod 81, a ratchet protrusion 87a, an inner annular ring 87b, and an Astra-type spool 85 may be added to the syringe 82 to provide single-handed control during aspiration and injection. The plunger rod 81 may be attached to a rubber plunger 89a as described above with respect to other embodiments. The spool 85 and the ratcheting protrusion 87a of the finger flange assembly snap together around syringe flanges 88 to hold the complete syringe assembly 80 together in a pre-armed state (see FIG. 8A). Once provided to the user, the finger flanges 84 and the spool 85 are squeezed together, engaging the annular ring 87b with the proximal end of the cartridge 83 and moving the cartridge distally into the syringe 82, causing the needle 86 to pierce the septum 89b, which arms the syringe assembly 80 for local anesthetic delivery (FIG. 8B). In the embodiment shown, in FIGS. 8A and 8B, the plunger rod has already been engaged with the cartridge plunger. In yet another embodiment, the plunger rod may not be initially engaged with the cartridge plunger. In this embodiment, the ratchet may be used to both engage the plunger rod and the cartridge plunger as well as to arm the syringe.

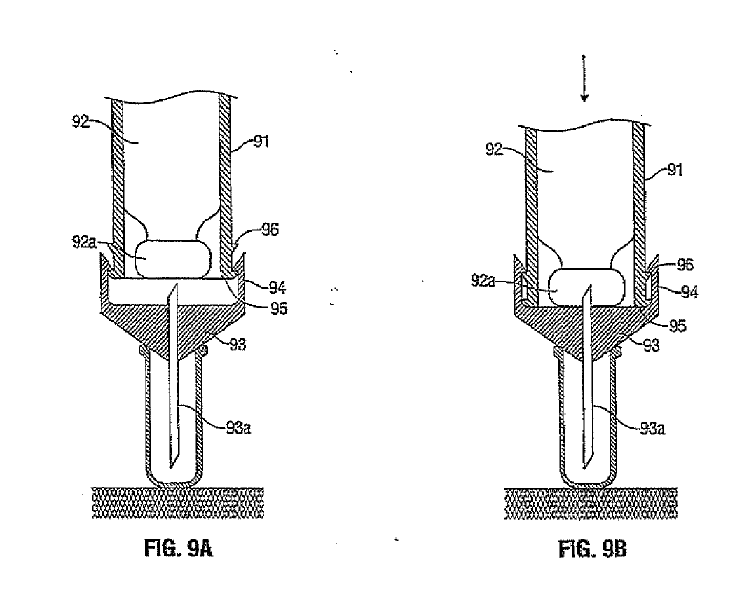

FIGS. 9A and 9B depict a needle hub 93 according to another embodiment of the present invention. A syringe 91 has a cartridge 92 loaded in a pre-armed state (see FIG. 9A). The needle hub 93 is attached to an outer diameter at a distal end of the syringe 91. The needle hub 93 includes a flange 94 that engages ratchet protrusions 95, 96 at the distal end of syringe 91. When the syringe/cartridge assembly is ready to be armed, the syringe 91 is pushed distally into the needle hub 93 until the cartridge 92 engages the needle hub 93, and the septum 92a is pierced by the needle 93a (FIG. 9B). When the syringe 91 is pushed distally, the ratchet protrusion 96 engages the flange 94 of the needle hub 93. The ratchet protrusion 96 prevents the syringe/cartridge assembly from being dislodged once armed. Alternatively, a threaded or a bayonet-style connector may be used to attach the syringe/cartridge assembly to the needle hub 93. One advantage of this embodiment is that one sleeve may receive a variety of sized and shaped cartridges because the hub cooperates with the sleeve to arm the cartridge. In another embodiment, not shown, an external surface of the hub cooperates with an internal surface of the sleeve, wherein a detent on an internal surface of the sleeve also engages the cartridge to prevent premature arming. This embodiment is also advantageous in that the sleeve may receive a variety of sized and shaped cartridges because the hub and the sleeve cooperate to arm the cartridge.

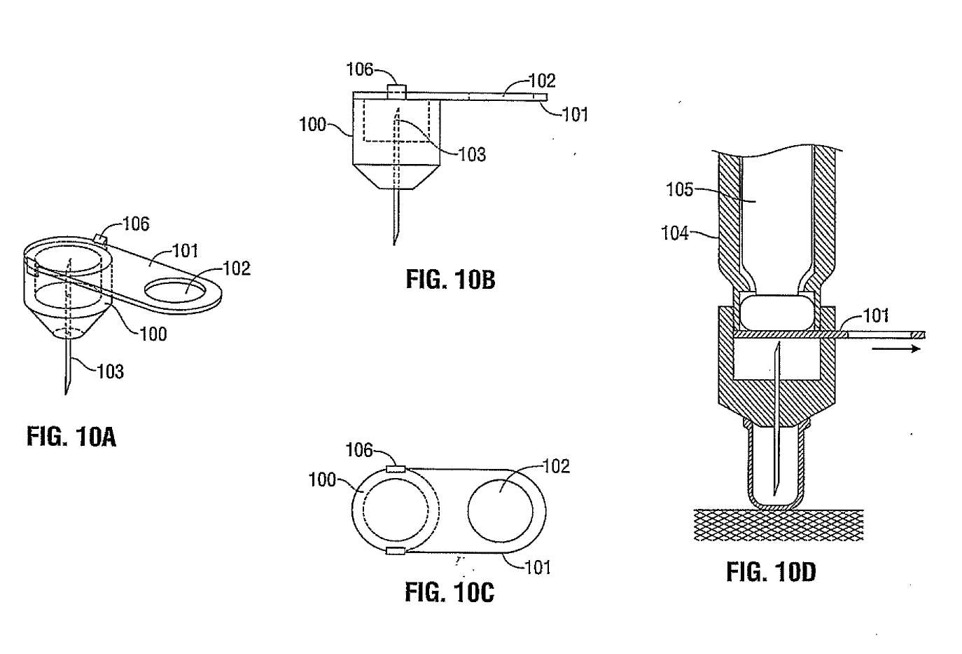

FIGS. 10A-10D depict a needle hub 100 having a needle 103 and a rigid plastic card 101 that acts as a sterile barrier and a mechanical stop to prevent inadvertently arming a cartridge 105 in a syringe or sleeve 104. The card 101 may have a large tab or loop 102 that is used as a handle to remove the card when the user is ready to arm the cartridge 105. The card 101 is kept in place by protrusions or flanges 106 formed integrally with the needle hub 100. Flanges 106 may also cooperate with a corresponding structure on the sleeve 104 to attach the needle hub 100 with the barrier to the sleeve. The card 101 may be completely or partially removed from the hub when ready to use. In one embodiment, the card is completely removed. In another embodiment the card is partially removed exposing the needle tip to the cartridge septum, but preventing an opening from forming which may allow contaminants to contact the cartridge.

In yet another embodiment, the cover may be a flexible sterile barrier made from foil or Tyvek sheet that is removed prior to attaching the transfer needle 31 to the cartridge 37. Tyvek is a US federally registered trademark of the E. I. du Pont de Nemours and Company. Alternatively, the sterile cover may remain in place while attaching the transfer needle 31 to the cartridge 37 so the needle 31 pierces the sterile cover. In another embodiment, the barrier may comprise two portions. A first portion may act as a barrier for the needle while the second portion may act as a barrier for the cartridge. The two portions may be individual barriers which may be simultaneously removed. Alternatively, the two portions may be integrally formed and removed in one motion. In yet another embodiment, the sterile barrier may by punctured during arming, thereby avoiding the need to remove the barrier prior to arming.

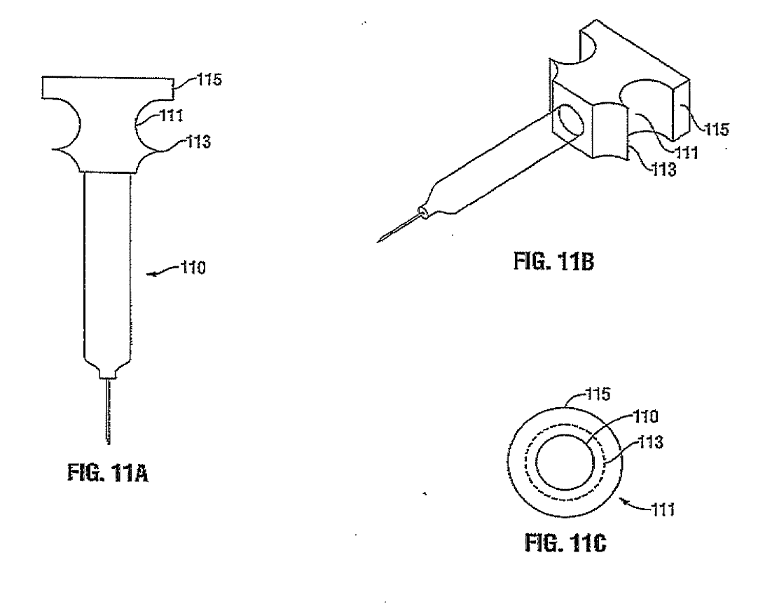

FIGS. 11A-11C depict a single use injection molded syringe having an integral finger flange assembly according to another embodiment of the present invention. FIG. 11A depicts a finger flange assembly 111 at the proximal end of a syringe 110. The finger flange assembly 111 includes a lower finger flange 113 and an upper finger flange 115. The lower finger flange 113 and the upper finger flange 115 are oriented radially outward perpendicular to a longitudinal axis of the syringe 110 to provide a clinician with control during aspiration. The lower finger flange 113 and the upper finger flange 115 may be oriented perpendicular to the longitudinal axis of the syringe 110 as shown in FIG. 11B or around the entire circumference of the syringe 110 as shown in FIG. 11C. One advantage embodiment shown in FIG. 11C is that extending the lower and upper finger flanges around the entire circumference of the syringe eliminates the need to orient the needle bevel to align with the flanges in order to introduce the needle into a subject at a desired angle.

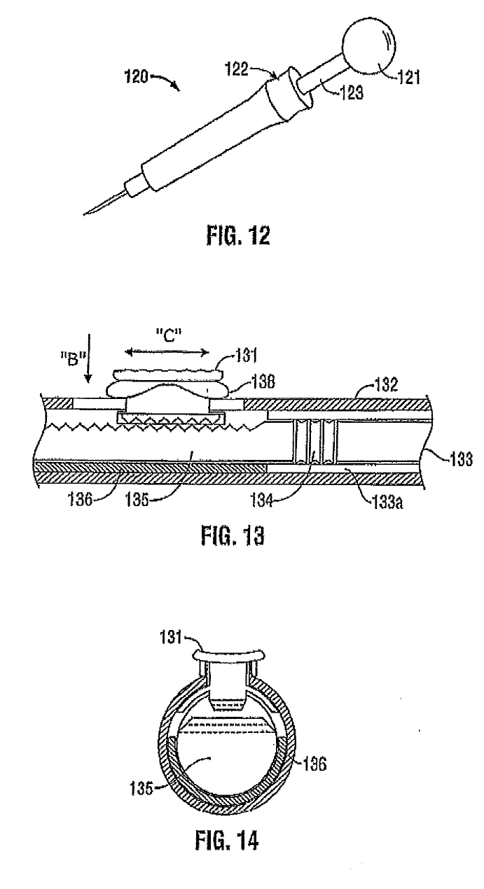

As illustrated in FIG. 12, a syringe 120 according to another embodiment of the present invention has a ball 121 at the proximal end of a plunger rod 123 instead of a ring. The ball 121 is placed in the palm of the user's hand while the user's fingers surround finger flange 122. This allows more accurate placement using the forefinger and leverage during injection and aspiration forces using the palm and thumb for accuracy. Although a ball is shown at the proximal end of the plunger rod 123, any solid mass that fits in the palm of a hand may be used.

FIGS. 13 and 14 depict a low profile design for a single use syringe according to another embodiment of the present invention. As shown in FIGS. 13 and 14, a sleeve 132 contains a cartridge 133 storing a liquid medicament such as a local anesthetic. A plunger 134 seals the medicament in the cartridge 133 and is used to aspirate or inject the anesthetic from the distal end of the syringe (not shown). The sleeve 132 includes a movable thumb slide 131 and a molded spring 138. The sleeve 132 also includes a plunger rod 135 threaded into the plunger 134 engaging the thumb slide 131 during aspiration or injection. A shim 136 is provided below the plunger rod 135 having the same thickness as a wall 133a of the cartridge 133. When the user wants to aspirate or inject the local anesthetic, the user presses down on the thumb slide 131 as shown by arrow “B” so the slide engages the plunger rod 135. The thumb slide 131 can now be moved proximally to aspirate or distally to inject medicament as shown by arrow “C”. The thumb slide may be engaged multiple times in one application to deliver the desired amount of medicament or to aspirate a sufficient amount of blood. In another embodiment, the thumb slide may be used with a prefilled syringe rather than a cartridge and sleeve configuration.

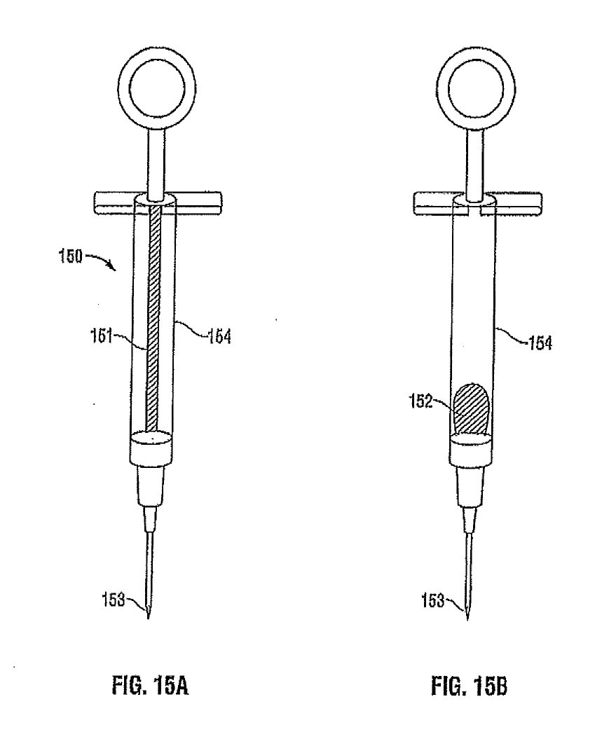

FIGS. 15A and 15B depict a single use syringe 150 in accordance with another embodiment of the present invention. The syringe 150 may be formed from a transparent or translucent material and may include a contrast strip 151 or a contrast mark 152 in the barrel 154. The contrast strip 151 and contrast mark 152 each provide a background to facilitate better visualization of blood within the syringe 150. This improves a user's ability to see blood in the syringe 150 against the background of a mouth or cavity. The contrast strip 151 or mark 152 can also serve as a visual indicator of the position of the needle bevel 153. The mark 152 may be extruded onto the barrel 154 or printed on an internal or external surface of the barrel 154. The strip 151 may be over laminated to secure the strip to the barrel 154. The strip 151 or mark 152 may be a light color or white to provide a contrast to see aspirate. In another embodiment, the contrast may be oriented with the bevel of a needle tip to quickly identify the proper orientation of the needle prior to insertion into a patient.

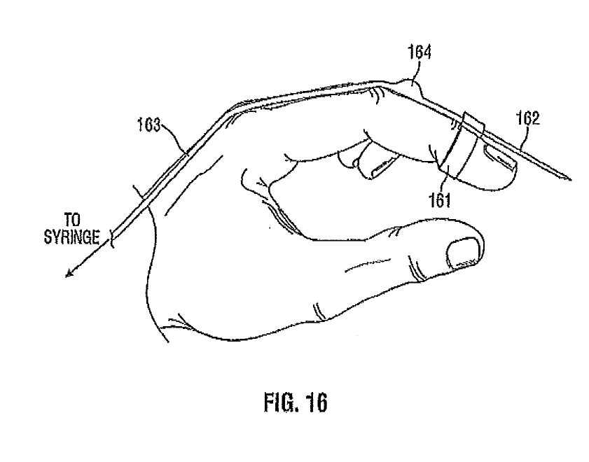

FIG. 16 depicts a needle assembly in accordance with another embodiment of the present invention. The needle assembly includes a finger engagement 161, such as a ring, worn on the user's finger. The finger engagement 161 includes a needle 162 that is fluidly coupled to a catheter 163 which is in fluid communication with a medicament reservoir (not shown). This allows a clinician to inject a medicament such as a local anesthetic into the subject, such as a patient's mouth, while making the procedure more comfortable for the patient because there is less hardware in the vicinity of the mouth. Because the medicament reservoir, such as a syringe, is remote from the needle, the clinician may be able to shield the syringe from the line of site of the patent which may reduce patient anxiety during a particular procedure. A balloon 164 may also be provided to measure the amount of medicament being injected. The presence of the balloon 164, which may fill or deflate with medicament during administration of the medicament may provide to normalize the delivery of medicament to the patent by temporarily inflating to accept a higher flow of medicament than desired or temporarily deflating to increase a lower flow than desired. Providing a smooth flow of medicament to the subject may reduce the pain associated with uneven and/or a high flow of medicament to an injection site. The balloon may also serve as an advancement indicator to warn the clinician that the needle has been inserted to an appropriate depth. In another embodiment, a protrusion extending from the needle may replace the balloon 161 as a depth indicator.

It should be noted that any of the features described above may be combined with one or more of any of the other features described in this application.

Although several embodiments of the disclosure have been shown in the drawings and/or discussed, those skilled in the art will appreciate the disclosure is not intended to be limited to the particular described embodiments, but should be read as broadly as the art will allow. Therefore, the above description should not be construed as limiting, but merely as exemplary. Those skilled in the art will envision other modifications within the scope and spirit of the claims appended hereto.

Having described the invention in detail, it will be apparent that modifications and variations are possible without departing from the scope of the invention defined in the appended claims.

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A syringe for administering medicament from a cartridge, the syringe comprising:
a sleeve having an interior sized and shaped for receiving the cartridge extending between a distal hub and a proximal opening opposite the hub sized and shaped for delivering the cartridge to the interior;
a cartridge having a medicament reservoir and a plunger, the cartridge disposed within the sleeve;
a cap permanently affixed to the sleeve and covering the proximal opening for retaining the cartridge in the interior of the sleeve, the cap including a finger grip extending outward from the sleeve for engaging fingers of a user administering medicament; and
a plunger rod movably connected to the cap, the plunger rod having a distal connector for connecting the rod to the cartridge plunger when the cartridge is received in the interior of the sleeve to push medicament from the cartridge reservoir as the plunger rod moves with respect to the cap.

2. The syringe of claim 1, wherein the plunger rod connector comprises a barb for engaging the cartridge plunger.

3. The syringe of claim 1, wherein the plunger rod connector comprises a threaded fastener for engaging the cartridge plunger.

4. The syringe of claim 1, further comprising a needle extending through the distal hub for piercing the cartridge to access medicament in the cartridge reservoir.

5. The syringe of claim 4, wherein needle is connected to the distal hub by a luer lock connector.

6. The syringe of claim 4, wherein the plunger rod is adapted to cover the needle before the syringe is used.

7. The syringe of claim 6, wherein the finger grip comprises a pair of finger rings extending outward from opposite sides of the cap.

8. The syringe of claim 6, wherein the plunger rod includes one of a proximal thumb ring sized for receiving a corresponding digit of the user and a ball.

9. A syringe for administering medicament from a cartridge, the syringe comprising:
a sleeve having an interior sized and shaped for receiving the cartridge extending between a distal hub and a proximal opening opposite the hub sized and shaped for delivering the cartridge to the interior;
a cartridge having a medicament reservoir and a plunger, the cartridge disposed within the sleeve;
a cap affixed to the sleeve and covering the proximal opening for retaining the cartridge in the interior of the sleeve, the cap including a finger grip extending outward from the sleeve for engaging fingers of a user administering medicament;
a plunger rod movably connected to the cap, the plunger rod having a distal connector for connecting the rod to the cartridge plunger when the cartridge is received in the interior of the sleeve to push medicament from the cartridge reservoir as the plunger rod moves with respect to the cap;
a catheter having a proximal end fluidly connected to the distal hub of the sleeve and a distal end fluidly connected to a needle.

10. The syringe of claim 9, further comprising a ring positioned on the second needle adapted to engage a user's finger.

11. The syringe of claim 10, wherein the needle further comprises a protrusion extending outwardly from a side of the needle.

12. A syringe for administering medicament from a cartridge, the syringe comprising:
a sleeve having an interior sized and shaped for receiving the cartridge extending between a distal hub and a proximal opening opposite the hub sized and shaped for delivering the cartridge to the interior;
a cartridge having a medicament reservoir and a plunger, the cartridge disposed within the sleeve;
a cap permanently affixed to the sleeve and covering the proximal opening for retaining the cartridge in the interior of the sleeve, the cap including a finger grip extending outward from the sleeve for engaging fingers of a user administering medicament; and
a plunger rod positioned over the needle and removably affixed to the distal hub of the sleeve, the plunger rod movably connected to the cap and having a distal connector for connecting the rod to the cartridge plunger when the cartridge is received in the interior of the sleeve to push medicament from the cartridge reservoir as the plunger rod moves with respect to the cap.

13. The syringe of claim 12, wherein the plunger rod is adapted to be removably affixed to the hub by a threaded connection.

14. The syringe of claim 13, wherein the plunger rod is adapted to be secured to the cartridge by a threaded connection.

15. The syringe of claim 12, wherein the sleeve includes a lock ring configured to engage the cartridge to prevent separation of the sleeve from the cartridge.

16. A syringe for administering medicament from a cartridge having a medicament reservoir and a plunger positioned adjacent reservoir for pushing medicament from the reservoir, the syringe comprising:
a sleeve having an interior sized and shaped for receiving the cartridge extending between a distal hub and a proximal opening opposite the hub sized and shaped for delivering the cartridge to the interior;
a cartridge disposed within the sleeve;
means for limiting movement of the cartridge towards the proximal opening of the sleeve;
a needle extending into the sleeve from the distal hub for piercing the cartridge to access medicament in the cartridge reservoir; and
a plunger rod movably connected to the sleeve, the plunger rod having a distal connector for connecting the rod to the cartridge plunger to push medicament from the cartridge reservoir as the plunger rod moves with respect to the sleeve, the plunger rod being adapted to move the plunger of the cartridge between a first position, in which the septum is not pierced by the needle and the plunger of the cartridge is in a proximal location, and a second position, in which the septum is pierced by the needle and the plunger of the cartridge is in a distal location.

17. The syringe of claim 16, wherein the syringe sleeve includes a detent located on an inner wall of the sleeve.

18. The syringe of claim 17, wherein the detent is adapted to space the cartridge from the needle to prevent the needle from penetrating the septum before the syringe prepared for use.

19. The syringe of claim 17, wherein the detent is adapted to engage an outer surface of the cartridge to prevent the cartridge from being dislodged.

20. A syringe for administering medicament from a cartridge having a medicament reservoir and a plunger positioned adjacent the reservoir for pushing medicament from the reservoir, the syringe comprising:
a sleeve having an interior sized and shaped for receiving the cartridge extending between a distal hub and a proximal opening opposite the hub sized and shaped for delivering the cartridge to and from the interior;
a needle extending from the distal hub for piercing a patient;
a flange adapted to engage a proximal end of the cartridge and a ratchet protrusion permitting the flange to move distally with respect to the sleeve and preventing the flange from moving proximally with respect to the sleeve so the ratchet protrusion retains the cartridge in a pre-armed state, in which the needle does not pierce the septum, before the flange is moved distally with respect to the sleeve and the ratchet protrusion prevents proximal movement of the flange with respect to the sleeve and retains the cartridge in an armed state, in which the needle pierces the septum, after the flange is moved distally with respect to the sleeve; and
a plunger rod movably connected to the flange, the plunger rod having a distal connector for selectively connecting the rod to the cartridge plunger when the cartridge is received in the interior of the sleeve to push medicament from the cartridge reservoir as the plunger rod moves with respect to the sleeve.

21. The syringe of claim 20, wherein the flange is an annular ring.

22. The syringe of claim 21, wherein the flange is permanently affixed to the sleeve.

23. The syringe of claim 21, wherein the flange is removably affixed to the sleeve.

24. A syringe for administering medicament from a cartridge having a medicament reservoir and a plunger reciprocally mounted in the reservoir for pushing medicament from the reservoir, the syringe comprising:
a sleeve having an interior sized and shaped for receiving the cartridge extending between a distal end and a proximal end opposite the distal end;
a plunger rod movably connected to the sleeve, the plunger rod having a distal connector for selectively connecting the rod to the cartridge plunger when the cartridge is received in the interior of the sleeve to push medicament from the cartridge reservoir as the plunger rod moves with respect to the sleeve; and
a hub moveably attached to the distal end of the sleeve, the hub including a needle extending toward the sleeve and being movable between a first position, in which the septum is not pierced by the needle, and a second position, in which the septum is pierced by the needle.

25. A syringe for administering medicament from a cartridge having a medicament reservoir and a plunger reciprocally mounted in the reservoir for pushing medicament from the reservoir, the syringe comprising:
a sleeve having an interior sized and shaped for receiving the cartridge extending between a distal hub and a proximal opening opposite the hub sized and shaped for delivering the cartridge to and from the interior;
a needle extending into the sleeve; and
a barrier positioned between the needle and the cartridge when received in the sleeve to prevent the needle from piercing the septum of the cartridge, wherein removal of the barrier permits the needle to pierce the septum to permit flow of medicament from the cartridge through the needle.

26. The syringe of claim 25, wherein the barrier is removably positioned between the needle and the cartridge.

27. A device for delivering medicament comprising:
a sleeve;
a medicament reservoir positioned within the sleeve;
a plunger positioned within the sleeve adjacent the medicament reservoir;
a plunger rod affixed to the plunger, wherein at least a portion of the plunger rod is positioned within the sleeve; and
a thumb slide movably attached to the sleeve and adapted to engage the plunger rod.
